(19) United States
(12) Patent Application Publication
(10) Pub. No.: US 2010/0036408 A1
(43) Pub. Date: Feb. 11, 2010

(54) RETRACTABLE LANCET AND RETRACTION MECHANISM FOR USE THEREWITH

(75) Inventor: Bradley S. Koeppel, Smyrna, GA (US)

Correspondence Address: GARDNER GROFF GREENWALD & VILLANUEVA. PC, 2018 POWERS FERRY ROAD, SUITE 800, ATLANTA, GA, 30339, US

(73) Assignee: Facet Technologies LLC, Kennesaw, GA (US)

(21) Appl. No.: 12/444,905

(22) PCT Filed: Oct. 26, 2007
(86) PCT No.: PCT/US07/82698

§ 371 Date: Apr. 9, 2009

Related U.S. Application Data
(60) Provisional application No. 60/862,998, filed on Oct. 26, 2006.

(51) Int. Cl. A61B 17/34

(52) U.S. Cl. 606/181

(57) ABSTRACT

A lancet for use with a lancing device, the lancet including a lancet body and a lancet needle. The lancet needle has a tip for piercing a subject's skin, and the tip of the lancet needle is retractable into the lancet body after use to prevent user contact with the now-contaminated tip. The needle retraction may be effected by a retraction mechanism including a control member with a cam surface and a ram surface and a lever with a cam follower and a needle engagement or stop member. As the control member is moved relative to the lancet, the cam follower transcribes the cam surface, which causes the engagement or stop member to retract the lancet.

### CROSS-REFERENCE TO RELATED APPLICATION

This application claims the priority benefit of U.S. Provisional Patent Application Ser. No. 60/862,998, filed Oct. 26, 2006, the entire scope and content of which is hereby incorporated herein by reference.

### TECHNICAL FIELD

The present invention relates generally to lancets for medical sampling of body fluids, and more particularly to lancets having retractable lancing tips for increased safety.

### BACKGROUND OF THE INVENTION

Lancing devices are used to penetrate the skin of a subject and obtain a sample of blood or other body fluid, as in the testing of blood sugar levels by diabetics. Typically, a lancet having a sharp point is translationally mounted within a lancing device. The lancet is driven by a spring or other biasing means to cause the sharp point to extend a small distance through an opening in the housing of the lancing device and into the subject's skin, creating a wound from which the sample of body fluid is collected. Lancing devices typically are intended either for a single use or for multiple uses.

Single-use lancing devices generally are disposed of after one use. For example, in a hospital or clinic, it is desirable to provide a single-use lancing device that can be used on a patient and then disposed of to eliminate any risk of infection to subsequent patients or caregivers from exposure to residual body fluids remaining on the lancing device. Accordingly, single-use lancing devices oftentimes include a disabling mechanism to prevent accidental or intentional re-use of the device. Various forms of disabling mechanisms are available, and are well known in the art. For example, the disabling mechanism may comprise a return spring for retracting the sharp point of the lancet back into the housing after a single use, break-away elements or a frangible link in the cocking or triggering mechanism to prevent re-arming or re-firing the device after a single use, a locking element, and/or a shield for blocking travel of the lancet.

However, multiple-use lancing devices generally permit a user to interchange used lancets with new lancets after each use. Once a lancet has been used, the tip is potentially contaminated with bloodborne pathogens, and should not be re-used. Typically, the used lancet is ejected or removed from the lancing device and a new lancet inserted therein. However, during removal of a used lancet from a lancing device, the needle of the lancet is typically exposed and can easily cause an unintended needle-stick to the user, a caregiver, or others. And, even after the lancet has been discarded, an exposed needle can unintentionally prick the skin of other people that may handle trash containing the discarded lancets. In order to minimize the risk of disease transmission, such unintended needle-sticks are to be avoided.

Accordingly, a need exists for lancets that reduce the likelihood of inadvertent contact with the sharp tip portions thereof. It is to the provision of lancets meeting this and other needs that the present invention is primarily directed.

### SUMMARY OF THE INVENTION

Generally described, the present invention is a lancet for use in collecting a sample of blood or other body fluid, the lancet including a lancet body and a lancet needle that is exposed for use to puncture skin and is not exposed at other times for safety. In example embodiments, the lancet needle has a tip for piercing a subject's skin, and after use the tip of the lancet needle is withdrawn to a retracted position within the lancet body where it is shielded from user contact. In addition, a retraction mechanism or other part of the lancing device may be provided to engage a portion of the lancet needle and cause the needle tip to be withdrawn to the retracted position after use. In other embodiments, a cap of the lancing device engages a portion of the lancet needle and pushes the lancet needle into the lancet body, whereby the lancet needle is concealed therein after use. An advantage of the lancets and retraction mechanisms of the present invention is that the sharp tip of the lancet needle is withdrawn to the retracted position within the lancet body after use, which prevents the now-contaminated lancet needle from injuring a user or other persons who may handle the lancet after it is ejected from the lancing device.

These and other aspects, features, and advantages of the invention will be understood with reference to the drawing figures and detailed description herein, and will be realized by means of the various elements and combinations particularly pointed out in the appended claims. It is to be understood that both the foregoing general description and the following brief description of the drawings and detailed description of the invention are exemplary and explanatory of preferred embodiments of the invention, and are not restrictive of the invention, as claimed.

### BRIEF DESCRIPTION OF THE DRAWINGS

FIG. 1A is a side view of a retractable-needle lancet according to a first example embodiment of the present invention.

FIG. 1B is a side view of a retractable-needle lancet according to a second example embodiment of the present invention.

FIG. 1C is a side view of a retractable-needle lancet according to a third example embodiment of the present invention.

FIG. 2A is a side view of the retractable-needle lancet of FIG. 1A, showing the lancet needle in the retracted position.

FIG. 2B is a side view of the retractable-needle lancet of FIG. 1B, showing the lancet needle in the retracted position.

FIG. 2C is a side view of the retractable-needle lancet of FIG. 1C, showing the lancet needle in the retracted position.

FIGS. 3A-3F show a sequence of operation of a retraction mechanism according to a fourth example embodiment of the invention in use with the retractable-needle lancet of FIGS. 1C and 2C.

FIGS. 4A-4F show a sequence of operation of a retraction mechanism according to fifth example embodiment of the invention in use with the retractable-needle lancet of FIGS. 1A and 2A.

### DETAILED DESCRIPTION OF EXAMPLE EMBODIMENTS

The present invention may be understood more readily by reference to the following detailed description of the invention taken in connection with the accompanying drawing figures, which form a part of this disclosure. It is to be understood that this invention is not limited to the specific devices, methods, conditions or parameters described and/or shown herein, and that the terminology used herein is for the purpose of describing particular embodiments by way of example only and is not intended to be limiting of the claimed invention. Also, as used in the specification including the appended claims, the singular forms “a,” “an,” and “the” include the plural, and reference to a particular numerical value includes at least that particular value, unless the context clearly dictates otherwise. Ranges may be expressed herein as from “about” or “approximately” one particular value and/or to “about” or “approximately” another particular value. When such a range is expressed, another embodiment includes from the one particular value and/or to the other particular value. Similarly, when values are expressed as approximations, by use of the antecedent “about,” it will be understood that the particular value forms another embodiment.

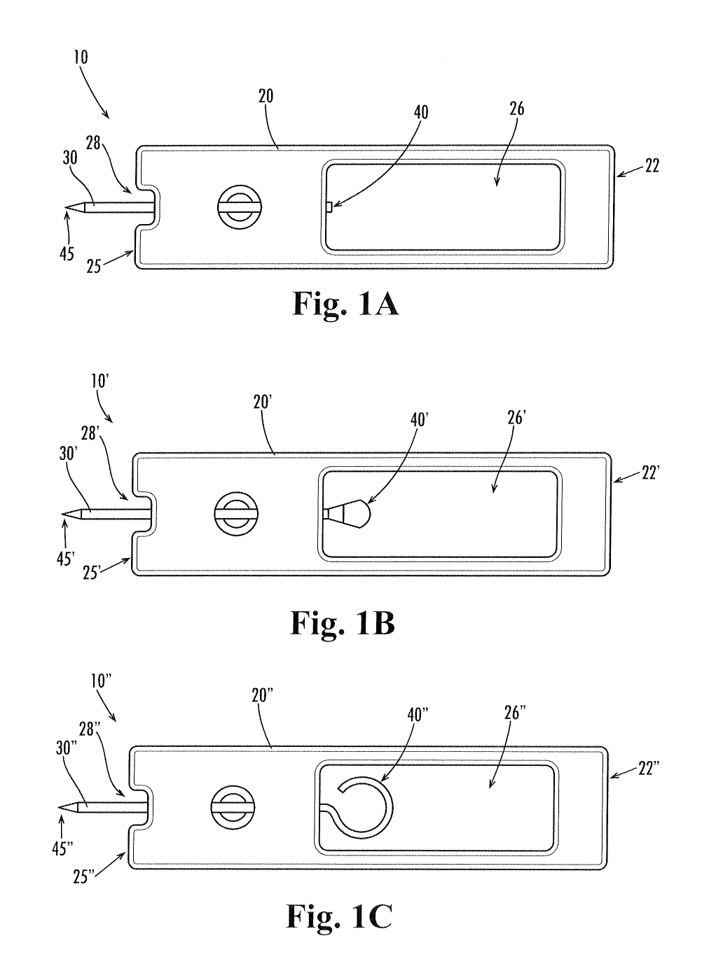

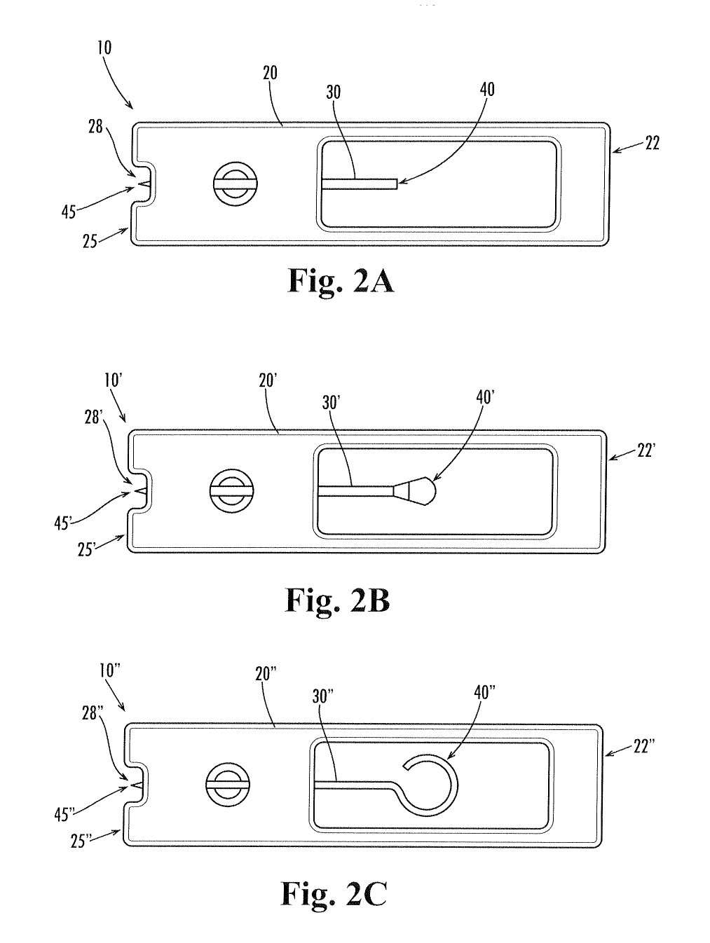

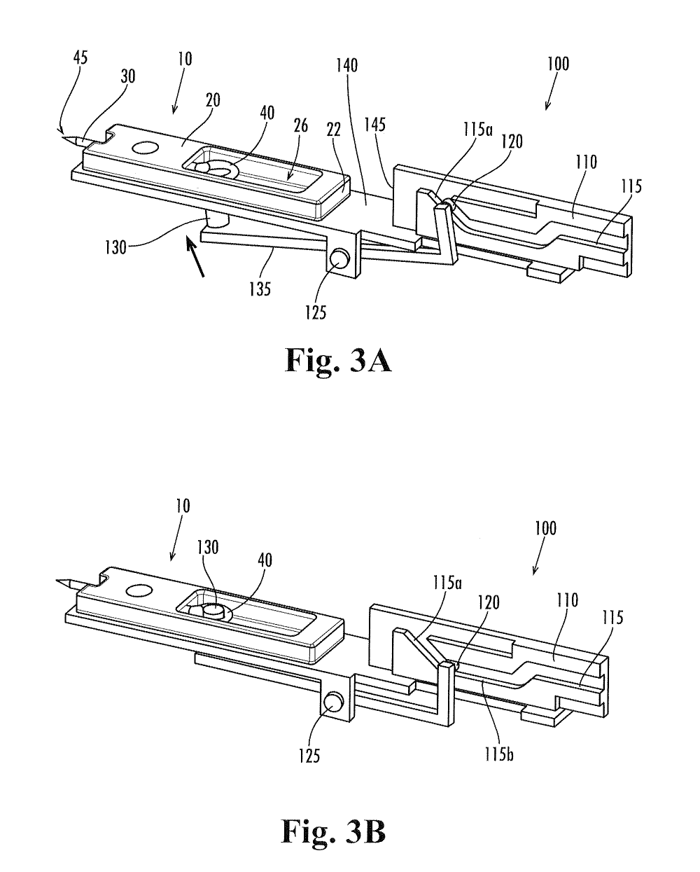

With reference now to the drawing figures, FIGS. 1A, 1B, and 1C show three retractable lancets 10 according to first, second, and third example embodiments of the present invention, respectively. Reference numbers for the second and third embodiments are the same as those for the first embodiment except they are annotated with prime and double-prime symbols, respectively. As used herein, the non-annotated reference numbers are meant to refer to all three embodiments collectively. Thus, the prime and double-prime symbols are used in the drawings figures but not in this detailed description.

The lancets 10 can be used in conjunction with a lancing device (not shown) to pierce a person's skin for extracting a sample of blood for testing purposes. Generally, the lancet 10 includes a lancet body 20 and a needle 30. As used herein, the term “needle” includes a blade or other sharp puncturing element. The lancet body 20 is preferably formed from plastic, although other materials can be used including rubber, metal, resin, composites, etc. The lancet needle 30 is preferably formed from metal, but can alternatively be constructed from other materials including ceramics and hard plastics.

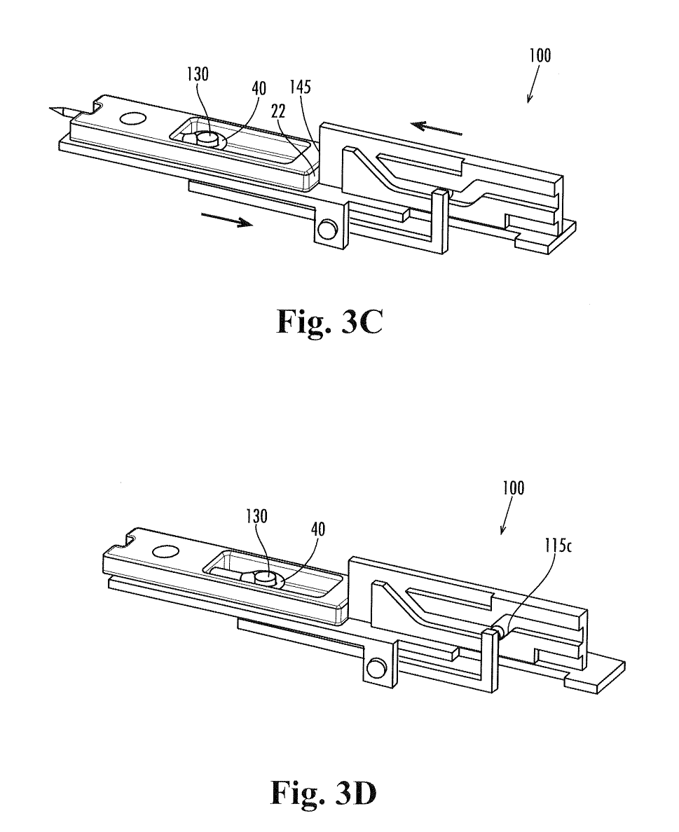

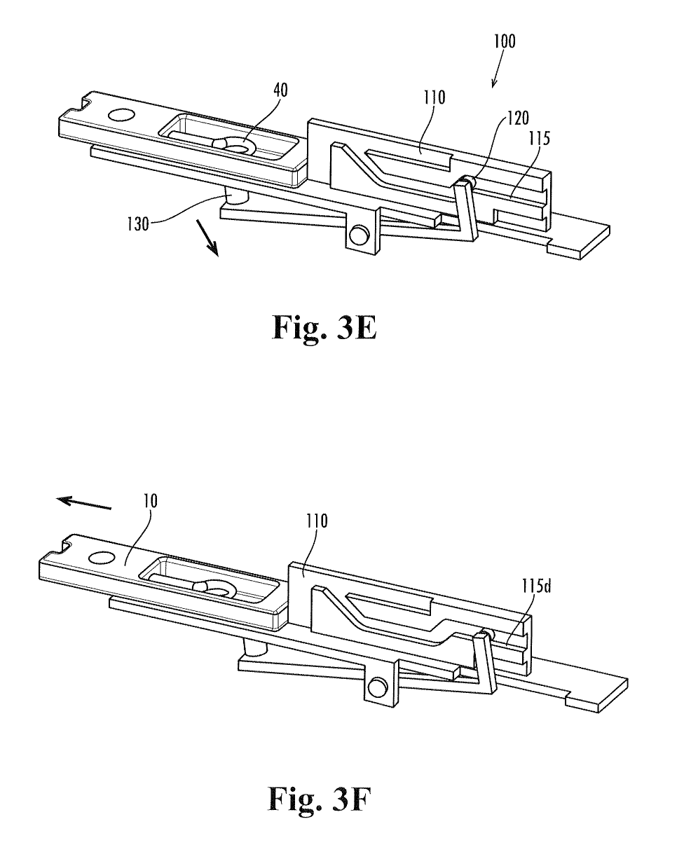

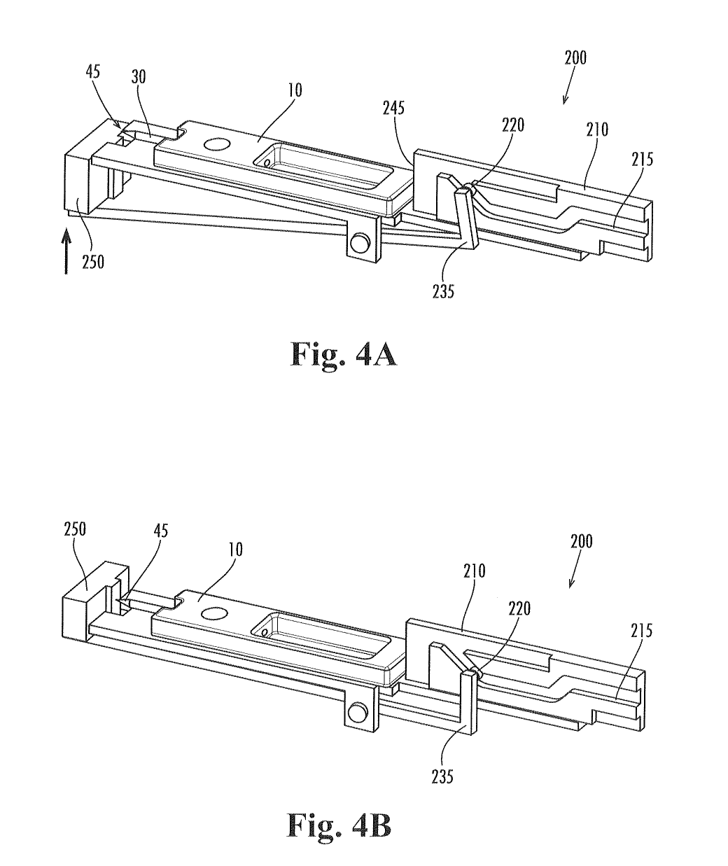

The lancet body 20 has a rear end 22 and a front end 25. The rear end 22 is typically received within a housing of the lancing device and driven by a spring- or otherwise-biased drive mechanism. The drive mechanism drives the lancet 10 axially along a drive path or stroke from a retracted position (see FIGS. 2A-2C) wherein the sharp tip is within the lancing device housing to an extended position (see FIGS. 1A-1C) wherein the tip extends a distance outside of the housing to penetrate the subject's skin to generate a sample of blood or interstitial fluid. In the extended position shown, the lancet needle 30 projects axially from the front end 25 of the lancet body 20 and extends rearward though a central bore (not shown) within the body. In the depicted example embodiments, the lancet body 20 is generally rectangular-shaped, having an axial length greater than its height or width. In alternate embodiments, the lancet body 20 is cylindrical, elliptical, polygonal, or otherwise configured, as determined by the particular application. In addition, in the depicted example embodiments the lancet body 20 defines an opening 26 that permits access to a rear end 40 of the lancet needle 30. The opening 26 of the depicted example embodiments is generally rectangular, however, the opening can be circular, elliptical, polygonal, notch-like, or otherwise configured. In alternate embodiments, the lancet body 20 has no such opening.

In the depicted example embodiments, the lancet needle 30 is generally cylindrical having a rear end 40 and a front end 45, although in alternate embodiments the needle is rectangular or polygonal in cross-section or otherwise shaped to meet the needs of a particular application. The front end 45 of the needle has a sharp point for piercing a subject's skin upon contact therewith. The rear end 40 of the lancet needle extends through at least a portion of the lancet body 20, as shown in the drawing figures, and preferably extends into the lancet body opening 26. In alternate embodiments, the rear end 40 of the lancet extends through the entire length of the body 20 such that the rear end 40 of the lancet extends through the rear end 22 of the lancet body. In the depicted example embodiments, the rear ends 40 of the lancet needles 30 are blunt, flared, or looped, as shown in FIGS. 1A, 1B, and 1C, respectively. In alternate embodiments, the rear end 40 is shaped and/or structured otherwise to cooperate with complementary retraction components (e.g., engagement or stop members) of a retraction mechanism or other component of the lancing device.

The lancet 10 of the present invention is unique in that the lancet needle 30 is retractable into the lancet body 20 itself after use to prevent user contact with the now-contaminated needle. After the lancet 10 has been used, the exposed needle 30 can be pulled or pushed back relative to the lancet body 20 so that the needle tip 45 is safely withdrawn to the retracted position as shown in FIGS. 2A-2C. In the depicted example embodiment, the lancet body 20 has a recess 28 (e.g., a notch or cutout) in its front end 25 into which the needle tip 45 is safely withdrawn in the retracted position, so that if the tip is not fully withdrawn into the body nevertheless the front end shields the tip in the recess from user contact. In alternate embodiments, the needle 30 is fully withdrawn into the body 20 of the lancet 10. In other forms of the invention, the needle is initially withdrawn to the retracted position within the lancet body, is extended after loading into a lancing device and used, and is withdrawn to the retracted position within the body 20 after use.

There are many options for effecting retraction of the tip 45 of the lancet needle 30 to the retracted position after use. The lancing device used to propel the lancet 10 into the skin of a subject can subsequently be used to withdraw the needle 30 after use. For example, the lancing device can be provided with a fork, hook, or other structure that is operable to engage the needle rear 40 and pull the needle through the lancet body 30 to the retracted position to conceal the tip 45. In other embodiments, the lancing device can be provided with a mechanism that is operable to push the lancet 10 into a plate or other solid structure after use to drive the lancet needle 30 into the body 20 of the lancet. For example, before a user can eject a used lancet 10, or during the actual process of ejecting the lancet, the lancet can be pressed against a hard plate or surface within the lancing device, or lancing device cap, to withdraw and conceal the needle 30. In still other embodiments, the manual or automatic rotation of a lancing device cap can conceal the lancet needle 30 within the body 20 of the lancet 10 by either pressing the tip 45 of the needle therewithin or by pulling the rear 40 of the needle to conceal the same. For example, a shutter or eccentrically located lancet opening can be provided in the endcap of the lancing device, such that after sampling is complete, the stroke of the lancet is blocked, and subsequent operation of the lancing device drives the tip 45 of the lancet needle into a hard surface causing retraction of the needle into the body 20.

In the depicted embodiments, the lancet needle 30 fits snugly within the central bore of the lancet body 20 such that sufficient friction exists between the needle and the bore sidewall to keep the needle stabilized during use. However, the frictional force between the needle 30 and the central bore sidewall is weak enough to permit the needle to be retracted into the lancet body 20 after use.

In another aspect of the invention there are provided retraction mechanisms that are operable to withdraw to a retracted position the tips of lancet needles of retractable-needle lancets such as those of the first, second, and third embodiments described above. In typical commercial embodiments, the retraction mechanisms are part of, or adapted to operate in conjunction with, an ejection mechanism of a reloadable lancing device. Conventional ejection mechanisms function to eject used lancets from lancing devices, and the retraction mechanisms are preferably adapted to retract the needle tips during the process of ejecting the used lancets. Such lancing devices and ejection mechanisms are well known in the art and therefore are not described in detail herein.

Generally described, the retraction mechanism includes a lancet needle engagement member, a cam follower operably coupled to the needle engagement member, and a control member with a cam surface. The needle engagement member is typically a needle end engager or a needle tip stop. When the control member is moved relative to the lancet body, the cam follower transcribes the cam surface, which manipulates the engagement member to engage and apply a force to the needle to withdraw the needle tip to the retracted position. In addition, the lancet body is preferably contacted and preventing from moving along with the lancet needle by a ram surface, which may be a part of the control member or a part another component of the lancing device.

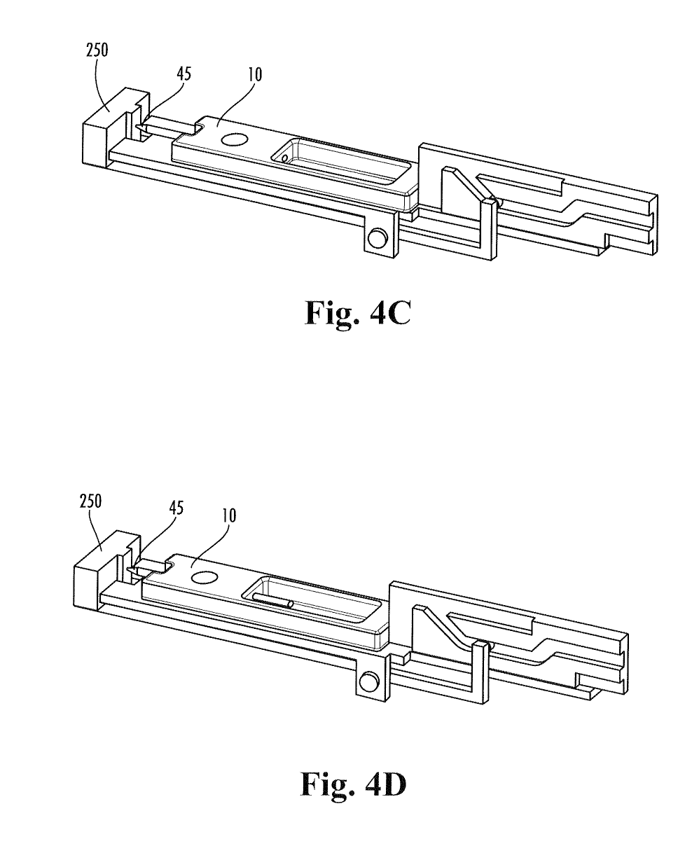

Referring to FIGS. 3A-3F, there is shown a retraction mechanism 100 according to a fourth example embodiment of the invention. The retraction mechanism 100 is particularly adapted to operate with the lancet of the third example embodiment, but it can be easily adapted to operate with other lancets with retractable needles. The retraction mechanism includes a translating control member 110 with a cam surface 115 and a lever 135 with a cam follower 120 and a lancet needle engager 130. The control member 110 moves relative to the lancet 10, for example it can be translationally carried along the top plane of a lancet holder 140, which supports the lancet 10 within the lancing device, and driven by a biasing mechanism of the lancing device. As the control member 110 is moved relative to the lancet 10, the cam follower 120 transcribes the cam path 115 and the needle engager 130 is thereby manipulated to engage, move, and disengage the lancet rear end 40 to pull the lancet tip 45 to the retracted position.

As shown in FIGS. 3A-3B, as the follower 120 moves along a first ramped segment 115a of the cam path 115, the lever 135 pivots about a fulcrum point 125 and the needle engager 130 is moved laterally (with respect to the axial lancing stroke path) into engagement with the needle rear 40 of the lancet 10. As shown in FIGS. 3B-3D, as the follower 120 moves along a second flat segment 115b of the cam path 115, the needle engager 130 is moved axially (along or parallel to the lancing stroke path) to thereby axially move the lancet needle 30 while a ram surface 145 of the control member 110 comes into engagement with ands applies force to the rear end 22 of the lancet 10. As such, the lancet needle 30 is moved in one direction and the lancet body 20 is moved in the opposite direction, which action causes the lancet needle tip 45 to withdraw to the retracted position of FIG. 3D (and FIG. 2C). As shown in FIGS. 3D-3E, as the follower 120 moves along a third ramped segment 115c of the cam path 115, after the lancet needle 30 has been withdrawn to the retracted position, the lever 135 return-pivots about the fulcrum point 125 and the needle engager 130 is moved laterally out of engagement with the needle rear 40 of the lancet 10. And as shown in FIG. 3F, as the follower 120 moves along a fourth flat segment 115d of the cam path 115, after the needle engager 130 has disengaged from the retracted lancet needle 30, the ram surface 145 axially moves the lancet 10 to eject it from the lancing device.

The depicted retraction mechanism 100 is adapted to work with the lancet 10 of FIGS. 1C and 2C. As such, the needle engager 30 is in the form of an elongated member (e.g., a pin, rod, post, arm, or finger) that is received within the looped rear end 40 of the needle 30. In alternative embodiments, the needle engager is in the form of a clasp, hook, yoke, catch, or other structure configured to engage and apply a pulling force on the blunt, flared, or otherwise-shaped rear end or other engagement component of the needle.

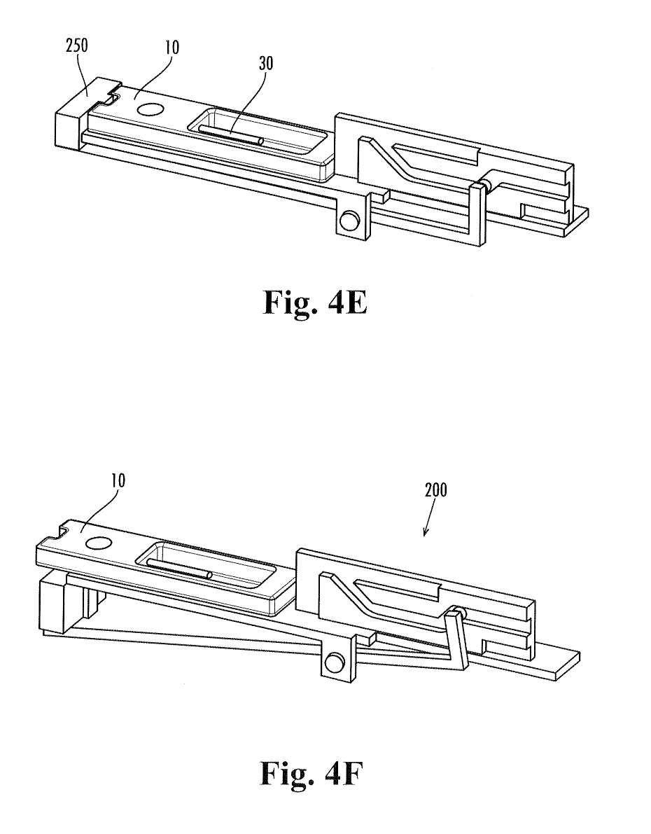

Referring to FIGS. 4A-4F, there is shown a retraction mechanism 200 according to a fifth example embodiment of the invention. The retraction mechanism 200 is similar to that of the fourth embodiment and includes a translating control member 210 with a cam surface 215 and a pivotal lever 235 with a cam follower 220, with the control member movable relative to the lancet. But instead of engaging and pulling the rear end of the lancet needle 30, the retraction mechanism 200 is operable to push the tip 45 of the lancet needle into the lancet body 20. To that end, the pivotal lever 235 has a lancet needle stop 250 at a distal end of thereof (instead of a lancet needle engager). As the control member 210 is moved relative to the lancet 10, the cam follower 220 transcribes the cam path 215 and the needle stop 250 is thereby manipulated to engage, move, and disengage the lancet needle tip 45 to push the lancet tip to the retracted position. In particular, as the cam follower 220 moves along the cam path 215, the stop 250 is raised into position in front of the lancet needle tip 45 (FIG. 4B), and as the lancet 10 is engaged and driven forward by the control member 210 (FIGS. 4C-4D), the needle tip 45 is pushed into the lancet body 20 (FIG. 4E). The lancet 10 is then ejected from the lancing device (FIG. 4F).

While the invention has been described with reference to preferred and example embodiments, it will be understood by those skilled in the art that a variety of modifications, additions and deletions are within the scope of the invention, as defined by the following claims.

What is claimed is:

1. A lancet for use with a lancing device to lance skin, the lancet comprising a lancet needle and a lancet body that holds the needle, wherein the lancet needle has a tip for piercing the skin during a lancing stroke, and wherein the tip of the lancet needle is retractable relative to the lancet body from an extended position projecting from the lancet body to a retracted position where it is shielded by the lancet body after use.

2. The lancet of claim 1, wherein the lancet needle is retractable in an axial direction relative to the lancing stroke.

3. The lancet of claim 1, wherein in the retracted position the lancet needle tip is positioned within the lancet body.

4. The lancet of claim 1, wherein the lancet body has a front end with a recess into which the lancet tip is withdrawn in the retracted position.

5. The lancet of claim 1, wherein the lancing device includes a retraction mechanism with a lancet needle engager, wherein the lancet needle has a rear end that extends from the lancet body and is adapted for engagement by the needle engager to withdraw the lancet tip to the retracted position.

6. The lancet of claim 5, wherein the lancet body has an opening through which the lancet needle rear end extends for access by the needle engager.

7. The lancet of claim 5, wherein the lancing device includes a retraction mechanism with a ram surface, and wherein the lancet body has a rear end that is adapted to be engaged by the ram surface to withdraw the lancet tip to the retracted position.

8. The lancet of claim 1, wherein the lancing device includes a retraction mechanism with a ram surface, and wherein the lancet body has a rear end that is adapted to be engaged by the ram surface to withdraw the lancet tip to the retracted position.

9. A retraction mechanism for use with a lancet having a lancet body and a lancet needle that is movable relative to the lancet body to a retracted position, the retraction mechanism comprising:
a control member having a cam surface, wherein the control member is translationally movable relative to the lancet body; and
a cam follower; and
a needle engagement member operably coupled to the cam follower,
wherein upon translational movement of the control member relative to the lancet body, the cam follower transcribes the cam surface, which manipulates the needle engagement member to engage the lancet needle and apply a force to the lancet to withdraw the needle tip the retracted position.

10. The retraction mechanism of claim 9, wherein the retraction mechanism further comprises a ram surface adapted to engage the lancet body to prevent the lancet body from moving along with the needle when the needle engagement member applies the force to the needle to withdraw the lancet tip to the retracted position.

11. The retraction mechanism of claim 10, wherein the lancet body has a rear end, wherein the ram surface is adapted to engage the rear end of the lancet body to prevent the lancet body from moving along with the needle when the needle engagement member applies the force to the needle to withdraw the lancet tip to the retracted position.

12. The retraction mechanism of claim 10 wherein the control member defines the ram surface.

13. The retraction mechanism of claim 9, further comprising a lever, wherein the cam follower and the needle engagement member extend from the lever.

14. The retraction mechanism of claim 13, wherein the lever is pivotally moveable.

15. The retraction mechanism of claim 9, wherein the lancet needle has a rear end, wherein needle engagement member is a needle engager that is adapted to engage and apply the force to the rear end of the needle to pull the needle tip to the retracted position.

16. The retraction mechanism of claim 9, wherein the lancet needle has a front tip, wherein needle engagement member is a needle stop that is adapted to engage and apply the force to the rear end of the needle to push the needle tip to the retracted position.

17. The retraction mechanism of claim 9, wherein the cam surface has at least one ramped segment and, when the cam follower transcribes the ramped segment, the needle engagement member is moved laterally into or from engagement with the needle.

18. The retraction mechanism of claim 9, wherein the cam surface has at least one flat segment and, in response to the cam follower transcribing the flat segment, the needle engagement member moves axially relative to the lancet needle to apply the retraction force to the lancet needle.

19. The retraction mechanism of claim 19, wherein the control member defines a ram surface that is adapted to engage and apply a force to the lancet body when the cam follower transcribes the second flat segment.

20. The retraction mechanism of claim 9, wherein the cam surface has a first ramped segment, a second flat segment, and a third ramped segment, wherein in response to the cam follower transcribing the first ramped segment the needle engagement member moves laterally into engagement with the needle, in response to the cam follower transcribing the second flat segment the needle engagement member moves axially relative to the lancet needle to apply the retraction force to the lancet needle, and in response to the cam follower transcribing the third ramped segment the needle engagement member moves laterally out of engagement with the needle.

21. The retraction mechanism of claim 19, wherein the cam surface has a fourth flat segment and, in response to the cam follower transcribing the fourth flat segment the needle engagement member is maintained out of engagement with the needle, and wherein the control member defines a ram surface that is adapted to engage and apply a force to the lancet body when the control member is moved relative to the lancet and the cam follower is transcribing the fourth flat segment, wherein the ram surface pushes the lancet body to eject the lancet after use.
